(19) United States
(12) Patent Application Publication
(10) Pub. No.: US 2008/0215089 A1
(43) Pub. Date: Sep. 4, 2008

(54) Stomach wall closure devices

(76) Inventors: Michael S. Williams, Santa Rosa, CA (US); Geoffrey A. Orth, Sebastopol, CA (US); Jeffrey A. Smith, Petaluma, CA (US); Richard A. Glenn, Santa Rosa, CA (US)

Correspondence Address: STALLMAN & POLLOCK LLP, 353 SACRAMENTO STREET, SUITE 2200, SAN FRANCISCO, CA, 94111, US

(21) Appl. No.: 11/903,340

(22) Filed: Sep. 21, 2007

Related U.S. Application Data
(60) Provisional application No. 60/826,535, filed on Sep. 21, 2006.

(51) Int. Cl. A61B 17/08

(52) U.S. Cl. 606/215

(57) ABSTRACT

In a method for sealing an incision in an interior body wall such as a gastrotomy opening in a stomach, a closure device is positioned within the incision. The closure device includes a seal and an anchor coupled to the seal. The seal is positioned in sealing contact against a first surface of the body wall, and the anchor is positioned against the second surface of the body wall such that a portion of the closure device is positioned. The closure device seals the incision while healing takes place. Once the incision is significantly healed, the closure device bioerodes.

### PRIORITY

This application claims the benefit of U.S. Provisional Application No. 60/826,535, filed Sep. 21, 2006.

### TECHNICAL FIELD OF THE INVENTION

The present invention relates generally to the field of natural orifice surgery, and more specifically to closure devices for closing incisions formed in the stomach wall to gain access to the peritoneal cavity.

### BACKGROUND

Systems and techniques in which access to the abdominal cavity is gained through a natural orifice are advantageous in that incisions through the skin and underlying muscle and peritoneal tissue may be avoided. Use of such systems can provide access to the peritoneal cavity using an access device inserted into the esophagus, stomach or intestine (via, for example, the mouth or rectum). Instruments are then advanced through the access device into the peritoneal cavity via an incision in the wall of the esophagus, stomach or intestine. Natural orifice access may also be gained by inserting instruments vaginally and forming an incision in the vagina or uterus to give access to pelvic organs or structures.

At the end of a natural orifice procedure, it may be desirable to close the incision formed in the stomach, intestine, uterus etc. The present application describes closure devices that may be used for this purpose, as well as systems and techniques for deploying the closure devices.

### BRIEF DESCRIPTION OF THE DRAWINGS

FIG. 1A is a front plan view of a first embodiment of a closure device.

FIG. 1B is a side elevation view of the closure device of FIG. 1A.

FIG. 1C is a perspective view of the closure device of FIG. 1A.

FIG. 1D is a top view of the closure device of FIG. 1A.

FIGS. 1E and 1F are a top view and a side elevation view of the closure device of FIG. 1A after each wing has been folded in preparation for insertion of the closure device into a delivery tube.

FIG. 1G is similar to FIG. 1F and shows the closure device following a second folding step.

FIG. 2A is a perspective view showing the closure device of FIG. 1A in a folded configuration and positioned next to a deployment system for use is placing the closure device in an abdominal wall incision.

FIGS. 2B through 2G are a sequence of perspective drawings illustrating deployment of the closure device of FIG. 1A using the FIG. 2A system.

FIG. 3 is a cross-section view of a portion of stomach wall and illustrates the closure device of FIG. 1A after it has been positioned as described in connection with FIGS. 2A through 2G.

FIG. 4A is a perspective view of a second embodiment of a closure device.

FIG. 4B shows a side elevation view of the closure device of FIG. 4A.

FIG. 5 is an exploded perspective view of a third embodiment of a closure device.

FIG. 6A is a side elevation view of a fourth embodiment of a closure device positioned in a stomach wall incision.

FIG. 6B shows the closure device of FIG. 6A positioned in a delivery cannula.

FIG. 6C shows the closure device of FIG. 6A deployed in a stomach wall incision.

FIG. 6D is a side view similar to FIG. 6B showing a modification to the FIG. 6A embodiment positioned in a delivery cannula.

FIG. 7A is a perspective view of a fifth embodiment of a closure device.

FIG. 7B is a side elevation view showing the FIG. 7A closure device positioned through an incision in a stomach wall.

FIGS. 8A and 8B are views similar to FIGS. 7A and 7B showing the closure device of the fifth embodiment during folding of the distal wing.

FIGS. 9A and 9B are views similar to FIGS. 7A and 7B showing the closure device of the fifth embodiment following folding of the distal wing.

FIG. 10A is a top plan view of a sixth embodiment of a closure device.

FIG. 10B is a side elevation view of the closure device of FIG. 10A.

FIG. 10C is a plan view similar to FIG. 10A showing the closure device, disposed in an incision through a stomach wall, following folding of the distal wing.

FIG. 11 is a perspective view showing a seventh embodiment of a closure device being inserted through an incision in a stomach wall.

FIG. 12A is a top plan view showing the closure device of FIG. 11 positioned in an incision.

FIG. 12B is similar to FIG. 12A and shows the closure device being collapsed to a folded position.

FIG. 12C is similar to FIG. 12B and shows the closure device in the folded position.

FIG. 13 is a perspective view of an eighth embodiment of a closure device.

FIG. 14 is a side elevation showing a ninth embodiment of a closure device positioned in an incision in a stomach wall.

FIG. 15 is a cross-sectional side view of a tenth embodiment of a closure device.

FIG. 16A is a perspective view of an eleventh embodiment of a closure device; FIG. 16B shows the closure device of FIG. 16A compressed into a delivery cannula.

FIG. 17A is a side elevation view of a twelfth embodiment of a closure device; FIG. 17B is a detail view of the region encircled by the arrows labeled 17B-17B in FIG. 17A; FIG. 17C shows the closure device of FIG. 17A positioned in a delivery cannula.

FIG. 18A is a side elevation view of a thirteenth embodiment of a closure device; FIG. 18B shows the device of FIG. 18A in a deflated state.

FIG. 19A is a perspective view of a fourteenth embodiment of a closure device; FIG. 19B is a side elevation view of the device of FIG. 19A.

FIG. 20A is a side perspective view of a fifteenth embodiment of a closure device positioned on a deployment mandrel; FIG. 20B shows the embodiment of FIG. 20A on the mandrel but in the deployed position.

FIG. 21A is a perspective view of a sixteenth embodiment of a closure device which utilizes separate attachable wings; FIG. 21B is a side elevation view of the closure device of FIG. 21A.

FIGS. 22A-22C are side elevation views showing alternatives to the separate attachable wing embodiment of FIG. 21A.

FIG. 23A illustrates a seventeenth embodiment of a closure device positioned within a delivery cannula; FIG. 23B is end view of the delivery cannula of FIG. 23A showing the closure device inside it.

FIGS. 24A through 24D are a sequence of steps illustrating deployment of the closure device of FIG. 23A.

FIG. 25A is a side elevation view of an eighteenth embodiment of a closure device; FIG. 25B is a side elevation view of the closure device of FIG. 25A in a delivery cannula.

### DETAILED DESCRIPTION

The present application describes a number of closure devices that may be endoscopically implanted (preferably in a transoral procedure) to close an incision or other type of opening or puncture in an interior body wall such as a stomach wall. For simplicity, any type of opening formed in the body wall will be referred to as an incision. The descriptions given herein will be described as a gastrotomy closure device for closing incisions formed in stomach walls, although the devices and associated methods are suitable for use in closing incisions in other body walls (e.g. the uterus, vagina, colon or other parts of the intestinal tract) as well.

In general, closure devices of the type described herein comprise a pair of expandable portions, one of which is positioned inside the stomach and the other of which is positioned on the stomach exterior. A connecting feature extends between the expandable portions and is generally positioned extending through the incision. The closure devices seal the incision preventing passage of fluids or material from stomach into the peritoneal cavity while the incision heals. They are preferably bioabsorbable/bioerodible implants so that they disappear once sufficient healing has taken place, but they may instead be permanent implants. In this disclosure, the term “bioerodible” will be used to describe any type of material that absorbs, degrades, erodes, etc. within the body over time. In some embodiments, the closure device additionally forms a platform or scaffold onto or through which tissue can grow during the healing process.

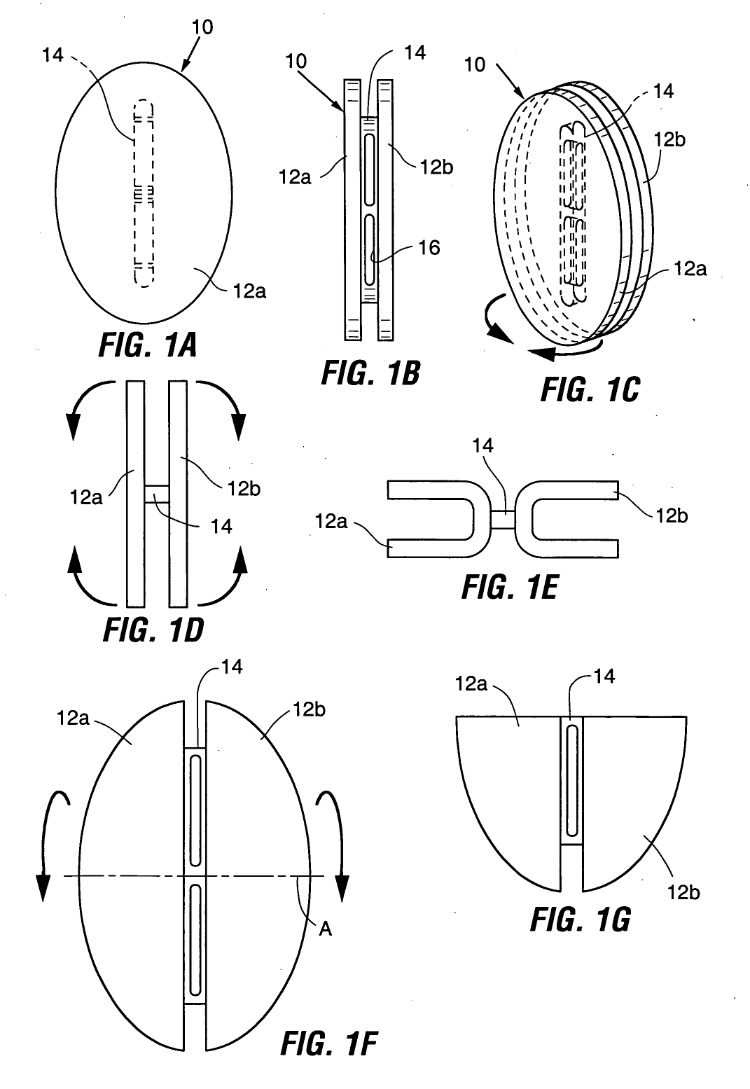

FIGS. 1A-1C illustrate a first embodiment of a closure device 10, which includes a pair of wings 12a, 12b and a connecting element 14 of any of a number of shapes extending between the wings. Wings 12a, 12b are shown as having an oval shape, although other shapes including, but not limited to, elliptical or circular shapes may be used. Also, the proximal wing 12b (or “interior” wing since it is placed in the stomach interior) may have a shape or configuration different from that of the distal (or “exterior”) wing 12a as described in the various embodiments discussed below.

In the first embodiment, the connecting element 14 is an elongate rib proportioned so that it may be positioned within an incision in the stomach. While not mandatory, the elongate shape of the rib is particularly suitable for a closure device used to close an elongate cut or tear in the tissue. The dimensions for the closure device are selected such that the spacing between the wings is sufficient to seal the incision without imparting excessive compressive forces on the stomach wall tissue. In one embodiment, the separation between the opposed surfaces of the wings is in the range of 0.06-0.1 inches.

The materials for the wings and rib are preferably materials that will bioerode, degrade or absorb after a period of time calculated to allow healing of the incision. Preferred materials include but are not limited to bioerodible elastomers or biorubbers such as those formed using sebacic acid materials. For some embodiments, non-woven bioerodible felts such as those made from polyglycolic acid fibers are particularly useful. Mesh, braid or woven materials formed using absorbable suture material may also be used. If mesh, braid or woven components are used for sealing components (e.g. one or both of the wings), they are desirably of sufficiently tight construction to prevent fluid passage through them, or the braid/mesh/suture is embedded in a bioerodible elastomer or biorubber, or they are sealed against fluid passage using bioabsorbable adhesives or other structures. The closure devices may be constructed with various combinations of materials. As one example, a device may have bioabsorbable polymer wings and a bioabsorbable mesh connector element. Additionally, each feature may have combinations of materials—such as a biopolymer reinforced by an embedded absorbable mesh structure. The materials may be coated or impregnated using sclerosing agents or other materials that will promote healing of the stomach wall tissue.

Ribs 14 may be provided with pores, openings or other features through which tissue may grow as the stomach tissue heals. In the FIG. 1A-1C embodiment, such features are in the form of slots 16.

The closure device 10 is constructed so it may be folded for insertion into a tube for deployment. Various folding arrangements may be used. One example is shown in FIGS. 1D-1F. FIG. 1D is a top view of the closure device prior to folding. As indicated by arrows, each wing 12a, 12b is first folded onto itself along its longitudinal axis, configuring the device 10 as shown in the top view of FIG. 1E and the side view of FIG. 1F. Next, with reference to FIG. 1F, the upper portion of the device 10 is folded across the horizontal axis A so that each wing 12a, 12b is again folded over on itself, placing the device 10 into the configuration shown in FIG. 1G.

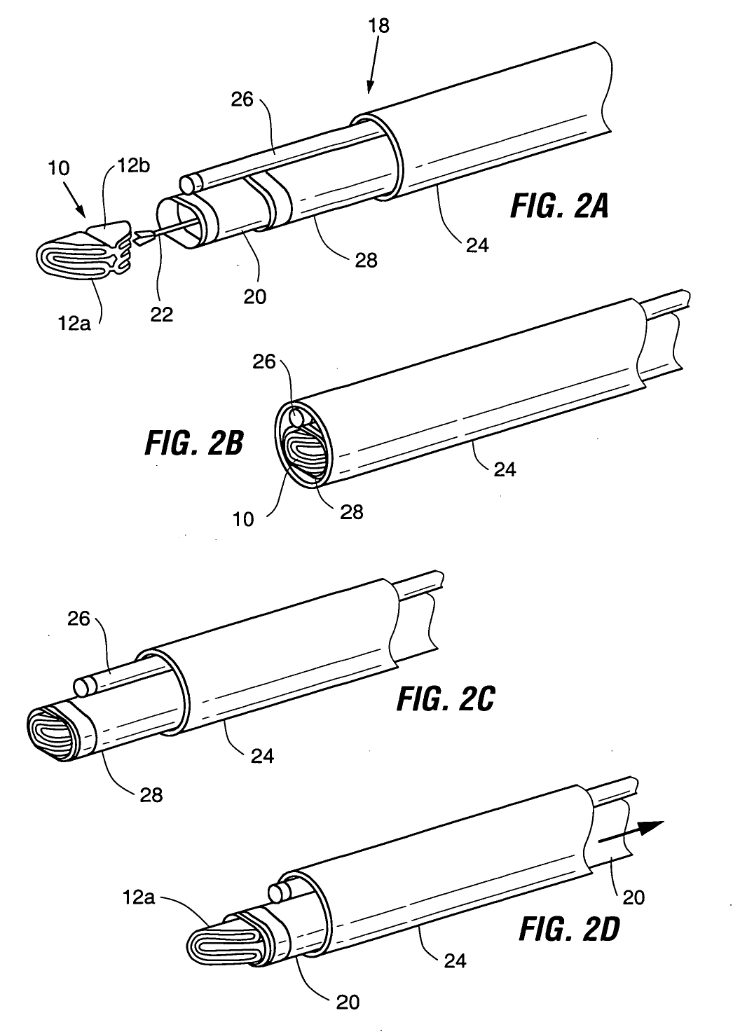

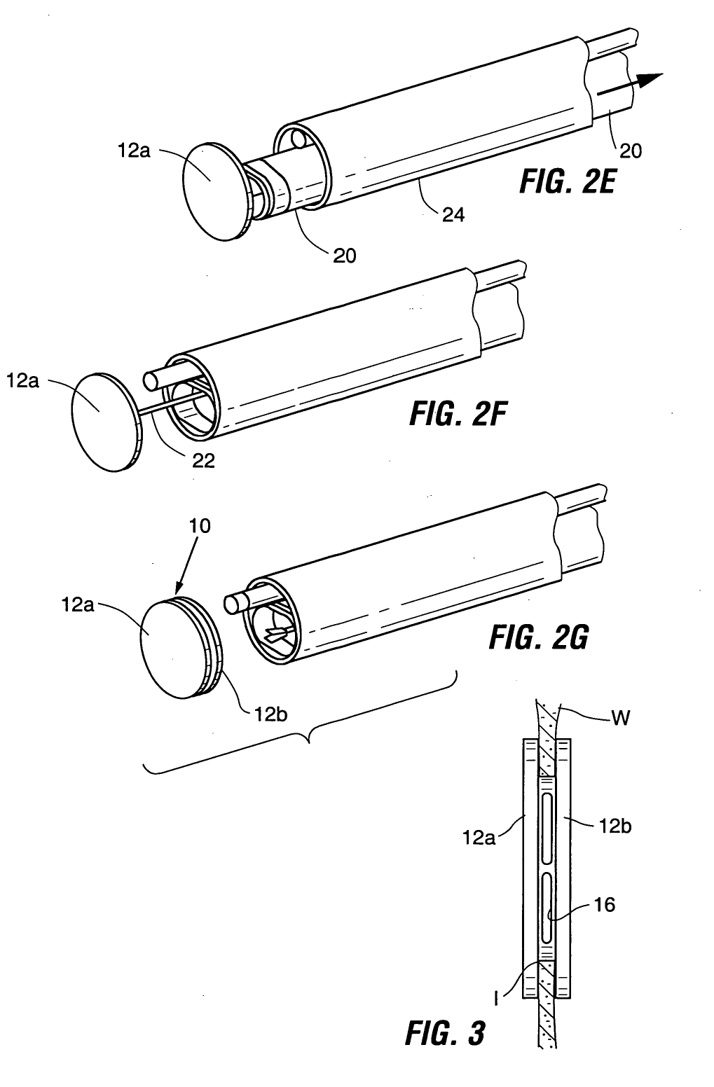

FIG. 2A illustrates a deployment system 18 of a type that may be used for implanting the closure device 10. System 18 includes a delivery cannula 20, a grasper 22 extending through cannula 20, a outer sheath 24, an endoscope 26 and an intermediate sheath 28. Use of the system 18 will next be described.

In preparation for deployment, the closure device 10 is folded as described above, and the proximal wing 12b to be deployed in the stomach interior is engaged in its folded state by grasper 22. The grasper 22 and a portion of the device 10 (including wing 12b) is withdrawn into the delivery cannula 20, leaving distal wing 12a positioned outside the distal opening of the delivery cannula 20. The delivery cannula 20 and the folded closure device 10 are positioned within the intermediate sheath 28 so as to maintain the folded configuration of the device 10. The intermediate sheath 28 and endoscope are positioned within the outer sheath 24 as shown in FIG. 2B.

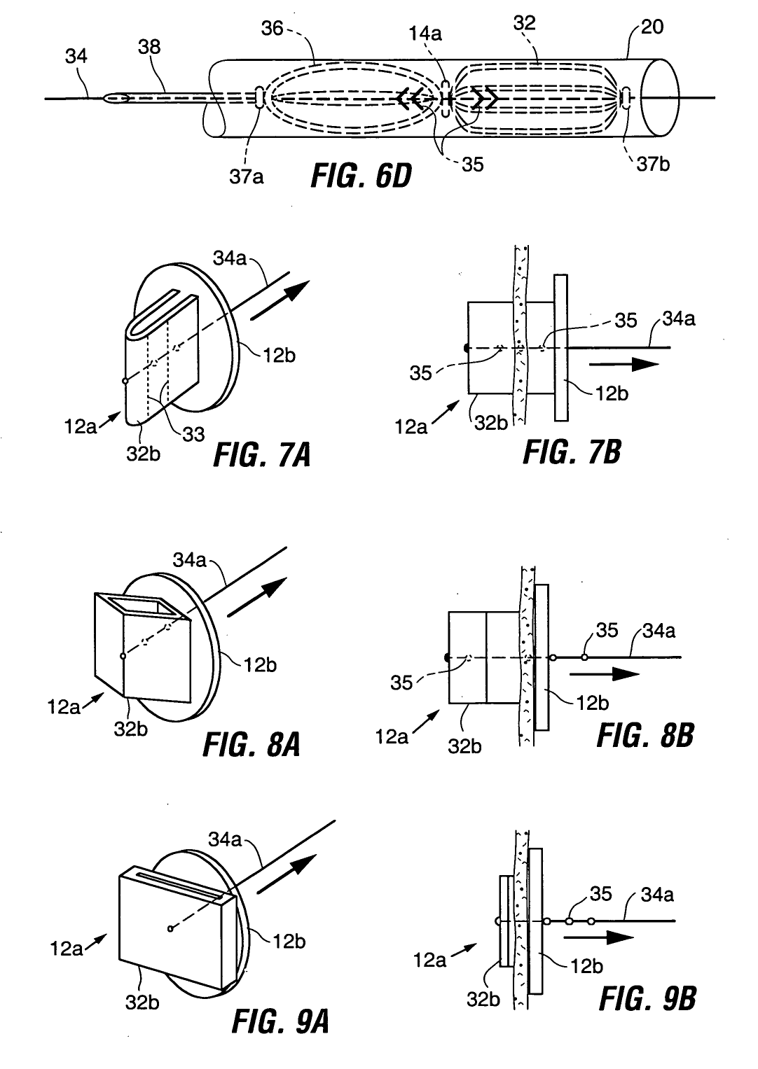

The distal end of the outer sheath 24 is passed through the mouth and esophagus and into the stomach. As shown in FIG. 2C, the intermediate sheath 28 is advanced out of the outer sheath 24 and through the incision (not shown) under visualization using the endoscope 26. At this stage the device 10 is within the intermediate sheath 28, along with the grasper 22 and delivery cannula 20, neither of which is visible in FIG. 2C. Referring to FIG. 2D, the intermediate sheath 28 is next withdrawn, exposing the exterior wing 12a of the device 10, causing the wing to expand on the exterior of the stomach to the position shown in FIG. 6. The delivery cannula 20 is withdrawn as shown in FIG. 2E, but the interior wing 12b remains folded because it remains within the jaws of the grasper 22. Traction is applied to the grasper to pull the exterior wing 12a into contact with the stomach wall. The grasper 22 is then actuated to release the wing 12b, causing it to expand in the stomach interior (FIG. 2F), leaving the device positioned within the incision as shown in FIG. 3. One or both of the wings 12a, 12b forms a seal with the stomach wall to prevent leakage of stomach contents into the peritoneal space. The elongate shape of the rib 14, which extends through the incision, helps to maintain the alignment of the sides of the incision. As the incision heals, tissue grows through the slots 16. Over time, the device degrades or absorbs within the body.

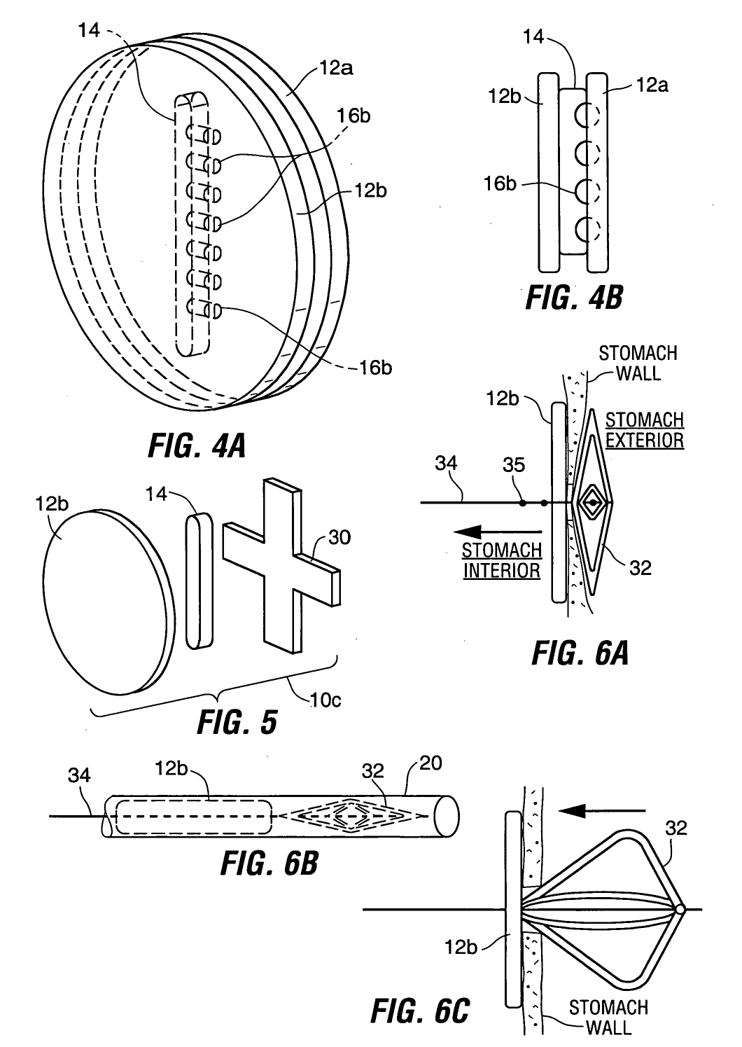

A second embodiment of a closure device 10b is shown in FIGS. 4A and 4B. Closure device 10b is similar to the first embodiment, except that the slots 16 of the first embodiment are replaced with a plurality of openings 16b in the rib 14. The openings 16b are positioned on the side of the rib closest to wing 12a. When the closure device is deployed, this configuration places the openings adjacent to the serosal tissue lining the exterior surface of the stomach. The openings thus create access through the device for serosal tissue bonding as serosal tissue grows through the openings from opposite sides of the incision. Serosal bonding is believed to be an important part of the stomach wall healing process.

As shown in FIG. 5, in a third embodiment of a closure device 10c, the exterior wing may be replaced with any feature that will expand on the exterior of the stomach to retain the closure device within the incision. Thus the illustrated embodiment includes an interior wing 12b and rib 14 which may be similar to the interior wing 12b and rib 14 of FIG. 1A, as well as a holder 30 formed of any shape (including but not limited to the illustrated X-shape) that will help to retain the closure device.

As another alternative shown in FIG. 6A, the exterior wing may be replaced with an anchor 32 formed, for example, using an arrangement of struts two or more. As shown in FIG. 6B, the anchor 32 is placed into delivery cannula 20 in a collapsed position. The distal end of the cannula 20 is passed through the incision in the stomach wall. Once the anchor 32 has been released from the cannula 20, it may be actively expanded by applying tension to a pull wire 34 (which is preferably a suture strand, but which might instead be a wire strand, rod etc) coupled to the anchor 32, causing the anchor to expand as shown in FIG. 6C.

Pullwire 34 may include knots or barbs 35 similar to those found on a zip tie fastener. The knots/barbs are used to engage the proximal/interior wing 12b (e.g. the material of the wing 12b or a collar, catch etc coupled to the interior wing 12b) to lock the anchor in the expanded position. In a variation shown in FIG. 6D, the interior wing 12b is a flexible tube that will form a disk when its ends are brought together. In this embodiment, the exterior anchor 32 is expanded by pulling the pull rod/wire 34, and the interior/proximal wing 36 is expanded by pushing distally on a mandrel 38 coupled to the proximal end of the tube forming wing 36. In the FIG. 6D embodiment, barbs/knots 35 on pullwire 34 engage collars 37a, 37b to lock the anchor 32a and wing 36 in the deployed positions.

In another embodiment shown in FIGS. 7A and 7B, the exterior (distal) wing 12a is formed of a strip 32b of material (e.g. a non-woven polyglycolic acid felt or other bioerodible material) doubled over on itself and coupled at its ends to the distal surface of proximal wing 12b. A pullwire (preferably length of suture 34a) is coupled to the apex of the strip 32b and extends through the proximal wing 12b. Fold lines 33, creases or thinned regions, are positioned in the strip 32b.

When tension is applied to the suture 34a, the strip 32b folds at the fold lines 33 as illustrated in FIGS. 8A and 8B. Folding preferably continues until the strip 32b collapses into a double layer wing as shown in FIGS. 9A and 9B. Knots 35 or other locking features on suture 34a contact the proximal surface of wing 12b, preventing reopening of the distal wing 12a.

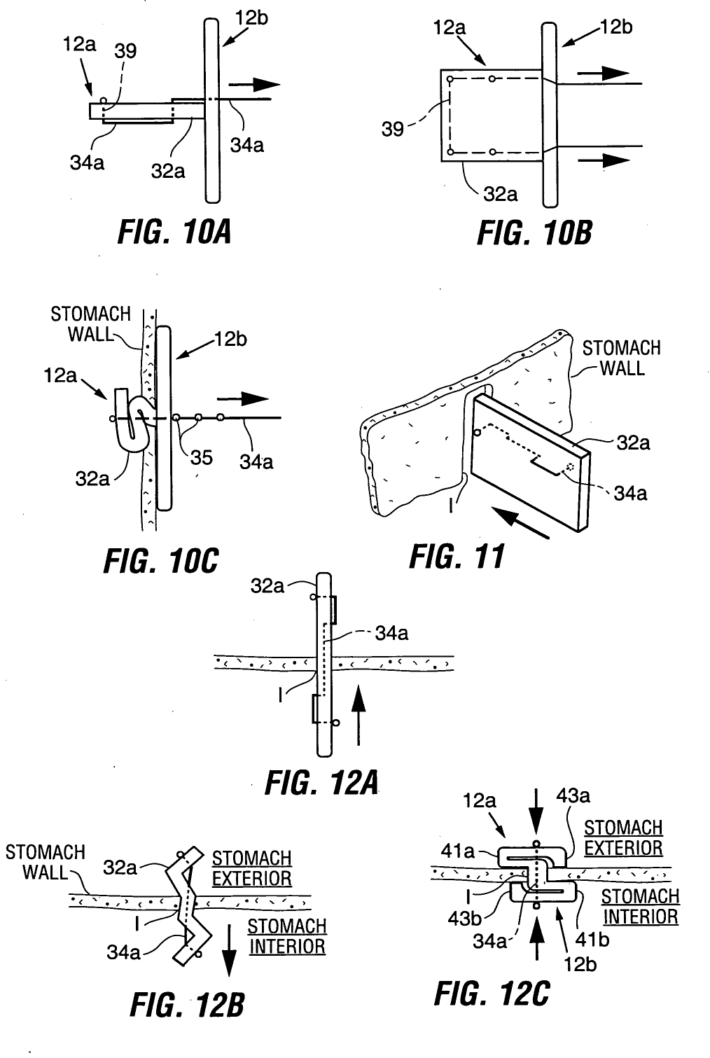

FIGS. 10A-10C illustrate an alternative to the FIG. 7A-9B embodiment, in which the distal wing 12a is also formed using strip 32a, but in which only one end of the strip 32a is coupled to the proximal wing 12b. In this embodiment, suture 34a is positioned to causes the strip 32a to fold to form the distal wing 12a when tension is applied to the suture 34a. For example, in the illustrated embodiment, suture 34a extends in a rectangular U-shaped pattern, with the lateral connector 39 of the “U” positioned near the distal end of the strip as shown in FIG. 10B. Each leg of the “U” extends along a first face of the strip 32a, then passes through the strip material and extends along the opposite face of the strip before passing again through the strip material.

To close an incision using the FIG. 10A-10C embodiment, the strip 32a is inserted through the incision and the proximal wing 12b is placed against the interior stomach wall as described above. When tension is applied to the end portions of the suture (see the arrows in FIGS. 10B and 10C), the strip 32a folds one or more times into a predetermined shape and seats against the exterior wall of the stomach, forming exterior wing 12a. For example, the FIG. 10A-10C embodiment is configured such that the lateral connector 39 of the “U” of the suture folds the distal end of the strip 32a into an orientation that is generally parallel the proximal wing 12b. As with the prior embodiments, the knots 35 engage with proximal wing 12b to lock the strip.

In the alternate embodiment of FIGS. 11-12C, strip 32a functions as both the proximal wing 12b and the distal wing 12a. As with the previous embodiment, a pullwire such as suture 34a is employed to collapse or fold the strip into a desired arrangement. The strip 32a is fed through the incision as illustrated in FIG. 11, and the suture is withdrawn to collapse the strip as shown in FIG. 12B. With the suture pattern shown in FIGS. 11-12C, full retraction of the suture places folds 41a, 41b in the strip 32a. The distal and proximal ends 43a, 43b of the strip preferably overlap the incision to facilitate sealing of the incision.

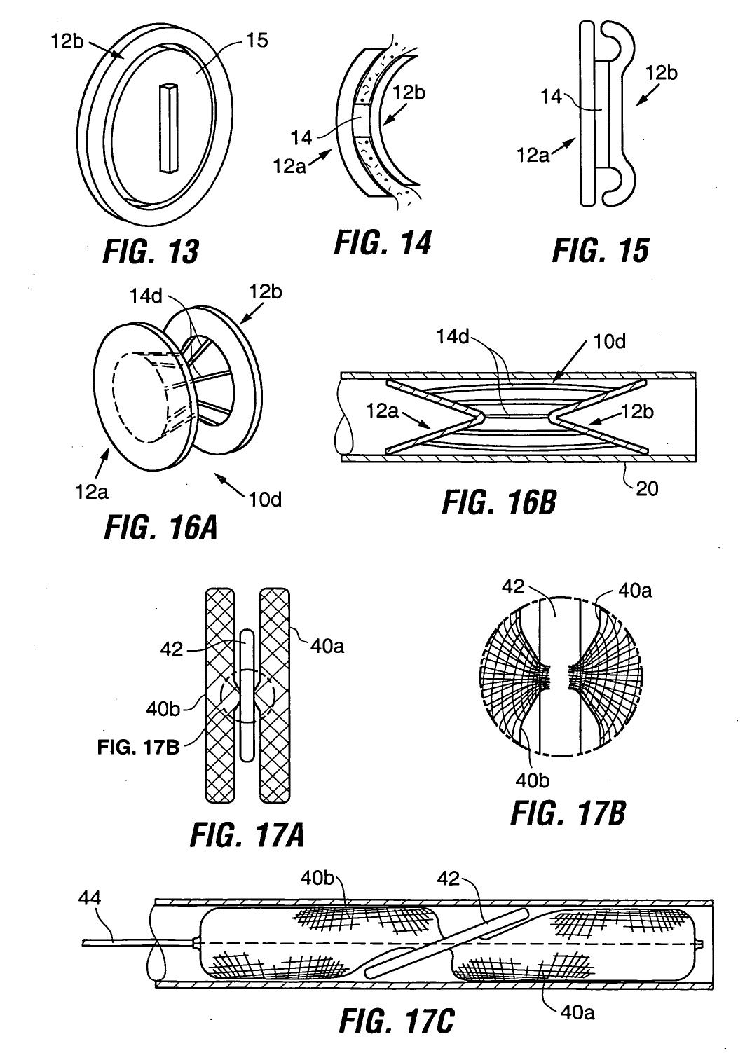

As illustrated in FIGS. 13-15, the closure device may be provided with features that facilitate sealing between the closure device and the stomach wall. For example, as shown in FIG. 13, inner wing 12b (or the outer wing if preferred) may include an annular seal 15 positioned to contact the stomach wall. The wings 12a, 12b may be contoured as shown in FIG. 13 to match the curvature of the stomach, or one or both of the wings may have edges shaped or biased such that they will lean into contact with the stomach wall as shown in FIG. 14.

In another closure device 10d shown in FIGS. 16A and 16B, the rib 14 of the FIG. 1A embodiment is replaced with a plurality of elastic ribs 14d extending between the wings 12a, 12b. This configuration may be fit into a delivery cannula 20 (FIG. 16B) by folding each wing 12a, 12b over on itself, causing the ribs 14d to stretch as shown. When the wing 12a is deployed from the cannula 20, the elasticity of the ribs pulls the wing 12a to its open positioned outside the stomach. The inner wing 12b likewise springs to its opened position when it is released from the cannula 20.

As discussed previously, any part or all parts of the closure devices described herein may be formed of a braid or mesh material. In one embodiment shown in FIGS. 17A and 17B, braided wings 40 are mounted to a rib 42 by molding ends of the braid material into the material of the center rib. In the illustrated embodiment, rib 42 has a rectangular or elongated oval shape and is formed of an elastic material. A mandrel 44 may be used as in FIG. 17C to advance the closure device from delivery cannula 20.

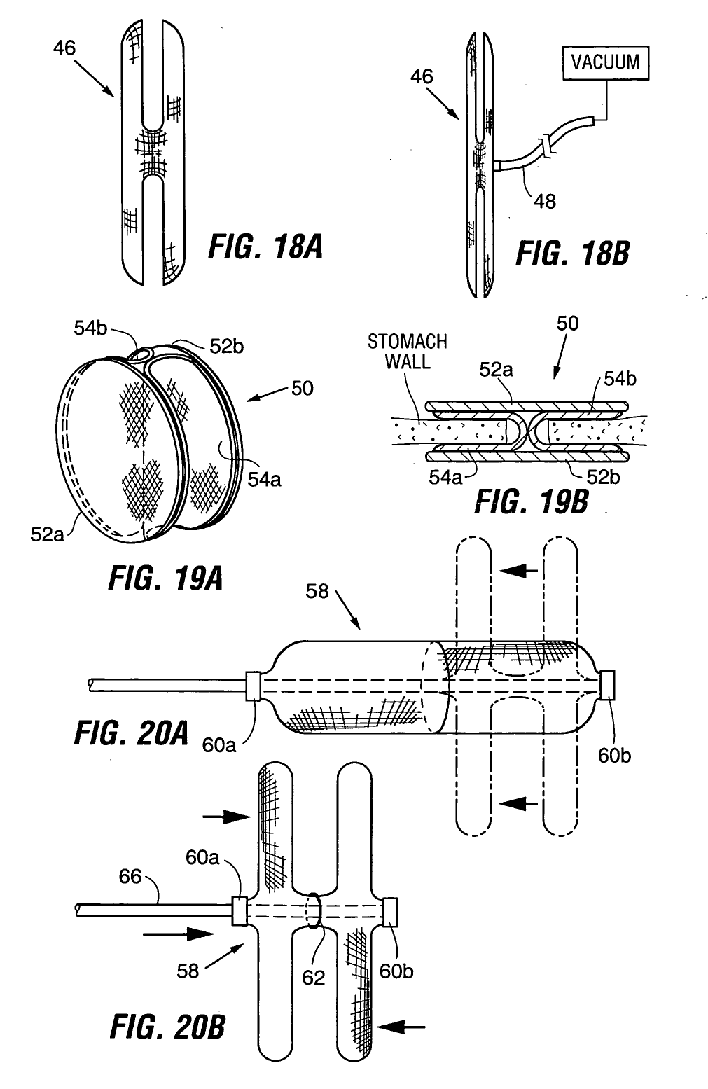

In another mesh/braid embodiment shown in FIGS. 18A and 18B, the closure device 46 is a hollow membrane formed of an absorbable mesh or thin film membrane, and may be filled with a “batting” material of polyglycolic acid (PGA) material or other absorbable biomaterial. A tube 46 is fluidly coupled to the interior of the device 46. Prior to implantation, a vacuum is applied to the tube 48 using a syringe or other vacuum source. Once positioned within the incision, saline or another fluid may be passed into the device 46 via tube 48 to expand the device. After filling, the tube 48 may be detached from the device 46 and removed from the body.

In the embodiment of FIGS. 19A and 19B, the device 50 is formed of four mesh disks 52a, 52b, 54a, 54b. Disks 52a, 52b form the wings of the device 50 and are preferably oriented in parallel to one another. Each of the disks 54a, 54b is connected to the wings 52a, 52b along its edges, so that when the wings 52a, 52b are deployed, the connector disks 54a, 54b contact one another as shown in FIG. 19B, forming a narrow connector that seats within the incision. A bioabsorbable glue may be used to couple the wings 52a, 52b to the connector disks 54a, 54b and/or to fill the interstices of one or more of the disks to prevent fluid migration through the incision.

In another embodiment shown in FIGS. 20A, 20B, the closure device 58 is a tubular braid having proximal and distal collars 60a, 60b. When expanded, the device 58 includes a narrow waist 62 formed by a restrictor band as shown, or by tapered construction of the braid material. The closure device is expanded by shortening the distance between the collars 60a, 60b using one of many techniques. Using the technique shown in FIG. 20bB, collar 60b is held in a fixed position on detachable mandrel 66 while collar 60a is advanced distally along the mandrel. The mandrel is detached following expansion.

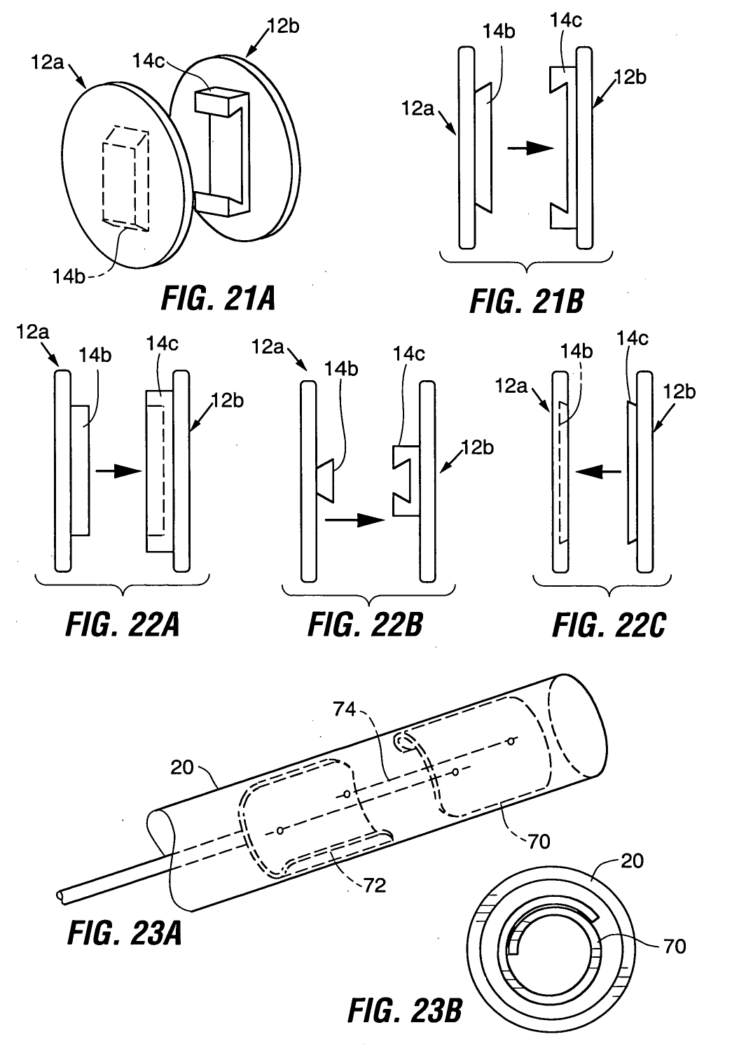

In alternative embodiments, a closure device similar to the closure device of FIG. 1A may have wings joined together using a rib formed of one or more pairs of interlocking pieces. Various configurations for interlocking ribs are shown in FIGS. 21A through 22C.

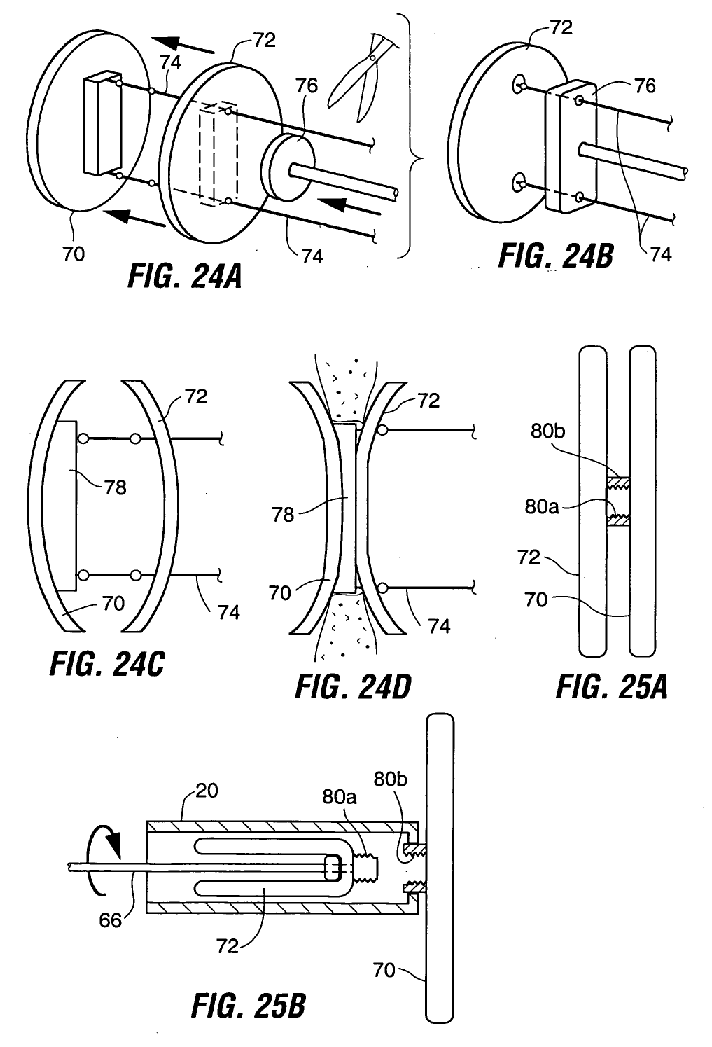

In another example of a two piece closure device illustrated in FIG. 24A, each of the wings 70, 72 may be threaded onto one or more barbed strands 74. With the wing 70 positioned outside the stomach and the strands extending through the incision, tension is applied to the strands as the wing 72 is pushed towards the stomach wall using a pusher 76. As illustrated in FIG. 24B, improved control over the pusher may be had by threading the pusher 76 onto the strands 74. As the wings 70, 72 are brought together, a rib 78 on one of the wings 70 extends through the incision and contacts the other wing 72. The rib 78 may slide into a corresponding recess 80 or interlocking feature in the wing 72. The barbed strands act as a “zip tie”, allowing the wings to be retained in a desired relationship relative to one another giving the user the ability to choose the amount of tissue compression to be used. Once the wings are positioned and the strands 74 tightened, the loose ends of the stands are clipped and removed from the body.

FIGS. 24C and 24D illustrate that the wings 70, 72 may be shaped such that when they are tightened against the tissue, their central regions bow inwardly to facilitate sealing of the incision. FIGS. 23A and 23B illustrate that the wings 70, 72, with the strands 74 coupled to them, may be positioned in a delivery cannula 20 for deployment.

FIGS. 25A and 25B illustrated an alternative two-piece design in which the wings 70, 72 are joined together within the body using a screw connection 80a, 80b.

In any of the disclosed the devices, sealing contact between the stomach wall and either or both of the proximal and distal wings/anchors may be enhanced through the use of adhesives. The adhesive may be a slowly degrading cyanoacrylate such as octyl-2-cyanoacrylate or N-butyl-cyanoacrylate. The adhesive may be applied onto the tissue surrounding the incision before the wing/anchor for that side of the tissue is placed. Any suitable applicators may be used for this purpose, including spray tips, sponges, syringes etc. If preferred, the wing/anchor may be itself be coated with adhesive, and a non-stick backing may be temporarily placed over the coating and then removed just prior to placement of the wing/anchor. Alternatively, microspheres filled with an adhesive may be attached to the wing/anchor and then punctured or broken prior to or during placement of the wing/anchor in contact with the tissue.

Any of the closure devices described herein may be packed as a system including delivery devices and/or instructions for use instructing the user to implant the closure devices according to methods disclosed herein.

Various components and methods have been described herein. These embodiments are given by way of example and are not intended to limit the scope of the present invention. It should be appreciated, moreover, that the various features of the embodiments that have been described may be combined in various ways to produce numerous additional embodiments. Also, while various materials, dimensions, shapes, implantation locations, etc. have been described for use with disclosed embodiments, others besides those disclosed may be utilized without exceeding the scope of the invention. For example, the devices are not limited to use within the stomach, but may be used to close incisions in other natural body cavities and elsewhere in the body.

Any and all patents, patent applications and printed publications referred to above, including those relied upon for purposes of priority, are incorporated by reference.

We claim:

1. A method for sealing an incision in an interior body wall, the method comprising:
identifying an incision in a body wall within a living body wall, the body wall having first and second surfaces;
positioning a closure device within the incision having a seal and an anchor coupled to the seal, positioning the closure device including positioning the seal against the first surface in a position covering the incision, and positioning the anchor against the second surface such that a portion of the closure device is positioned extending through the incision.

2. The method according to claim 1, wherein the method includes placing the closure device within a cannula, and wherein positioning the closure device includes advancing a distal end of the cannula through the incision, advancing one of the anchor and the seal from the cannula, withdrawing the distal end of the cannula through the incision and advancing the other of the seal and the anchor from the cannula.

3. The method according to claim 1, wherein position the anchor against the second surface causes the anchor to seal against the second surface in a position covering the incision.

4. The method according to claim 1, wherein the closure device degrades following healing of the incision.

5. The method according to claim 1, wherein the body tissue grows through at least a portion of the closure device during healing of the incision.

6. The method according to claim 1, wherein the body wall is selected from a group of body walls consisting of a stomach wall, intestinal wall, and uterine wall.

7. The method according to claim 1, wherein positioning the anchor includes passing the anchor through the incision and then expanding the anchor to anchor the closure device within the incision.

8. The method of claim 7, including passing the anchor in an elongated position through the incision, and longitudinally compressing the anchor to cause lateral expansion of the anchor.

9. The method of claim 1, wherein the closure device includes a member having a first portion coupled to the seal and a second portion coupled to the anchor, and wherein the method includes attaching the first and second portions.

10. The method of claim 9, wherein the first and second portions are coupled after the anchor is positioned.

11. A closure system for an interior body wall incision, the device comprising:
a seal positionable on a first side of a body wall in sealing contact with an incision through the body wall;
an anchor coupled to the seal and positionable on the second side of the body wall opposite the first side, the anchor expandable to engage tissue on the second side to retain the closure device within the incision.

12. The closure system of claim 11, wherein the anchor is bioerodible.

13. The closure system of claim 11, wherein the seal is bioerodible.

14. The closure system of claim 11, including a connector between the seal and anchor, the connector extending through the incision when the seal is positioned on the first side and the anchor is positioned on the second side.

15. The closure system of claim 14, wherein the connector includes a first portion on the seal and a second portion on the anchor, the first and second portions engageable to one another.

16. The closure system of claim 11, including openings in the connector positioned to received tissue ingrowth from the body wall.

17. The closure system of claim 11, wherein the anchor includes a strip coupled to the seal, the strip extendable through the incision, and an element coupled to the strip, the strip compressible upon application of tension to the element to cause folding of the strip.

18. The closure system of claim 16, wherein the strip includes a distal portion and a proximal portion, the strip positionable with the distal portion adjacent the second side and the proximal portion adjacent the first side, the strip compressible upon application of tension to the element to cause folding of the distal portion to form the anchor, and to cause folding of the proximal portion to form the seal.

19. The closure system of claim 11, further including instructions for use instructing a user to position the seal against the first surface in a position covering the incision and to position the anchor against the second surface.

20. The closure system of claim 11, wherein the instructions further instruct the user to adhere the seal to the first surface using an adhesive.

21. The closure system of claim 11, further including a delivery cannula of sufficient length to extend through an oral cavity into a stomach having the incision.
